(19) United States
(12) Patent Application Publication
(10) Pub. No.: US 2002/0164620 A1
(43) Pub. Date: Nov. 7, 2002

(54) Method for identifying compounds modulating sister chromatid separation

(75) Inventors: Jan-Michael Peters, Korneuburg (AT); Irene Waizenegger, Vienna (AT); Wolfgang Sommergruber, Purkersdorf (AT)

Correspondence Address: STERNE, KESSLER, GOLDSTEIN & FOX PLLC, 1100 NEW YORK AVENUE, N.W., SUITE 600, WASHINGTON, DC, 20005-3934, US

(73) Assignee: Boehringer Ingelheim International GmbH

(21) Appl. No.: 10/051,311

(22) Filed: Jan. 22, 2002

Related U.S. Application Data
(60) Provisional application No. 60/297,440, filed on Jun. 13, 2001.

(30) Foreign Application Priority Data
Jan. 19, 2001 (EP) 01 101 252.3

(51) Int. Cl. C12Q 1/68; G01N 33/567

(52) U.S. Cl. 435/6; 435/7.21

(57) ABSTRACT

Screening methods for identifying separase inhibitors based on active forms of separase and compounds identified in such methods.

The present application claims the benefit, under 35 U.S.C. § 119, of the earlier filing dates of European Patent Application No. EP 01 101 252.3, filed Jan. 19, 2001, and U.S. Provisional Application No. 60/297,440, filed Jun. 13, 2001. The contents of each of these applications are entirely incorporated herein by reference.

The invention relates to compounds influencing mitosis and meiosis in eukaryotic cells and methods for identifying such compounds. In particular, the invention largely relates to the treatment and prevention of human conditions by modulating sister chromatid segregation.

A key prerequisite for the successful division of one cell into to two is the duplication and subsequent segregation of the cellular genome into the two forming daughter cells. Duplication of the genome by DNA replication occurs during synthesis (S) phase of the cell cycle, whereas segregation of the duplicated DNA takes place much later during anaphase of mitosis. During S phase, replicated DNA molecules remain physically attached to each other, a phenomenon called cohesion, until they are separated in anaphase. At the beginning of mitosis, the cellular DNA is condensed into chromosomes, in which each of the two replicated DNA molecules are microscopically visible as sister chromatids. To allow the segregation of sister chromatids to the forming daughter cells in anaphase, the cohesion that is holding sisters together has to be dissolved. This process is mediated by a protease, called separin or separase, that is cleaving a complex of chromosomal cohesion proteins (the cohesin complex) that is required to hold sister chromatids together. This cleavage reaction liberates sisters from each other so that they can be pulled towards opposite poles of the diving cells by the spindle apparatus (reviewed by Nasmyth et al., 2000).

Inhibition of cohesin cleavage by separase in experimental systems such as budding yeast or human cultured cells inhibits sister chromatid separation and thus prevents the formation of viable daughter cells (Uhlmann et al., 1999; Uhlmann et al., 2000; Hauf et al., 2001).

WO 00/48627 suggests a method for identifying compounds which exert their effect by directly modulating, in particular by inhibiting separase's proteolytic activity, i.e. by being protease inhibitors specific for separase. (In the following, if not otherwise stated, “separase” stands for “human separase”.)

The screening method for identifying compounds that have the ability of modulating sister chromatid separation in plant or animal cells as described in WO00/48627 comprises incubating separase, in the presence of the substrate(s) for its proteolytic activity and optionally its co-factor(s), with test compounds and determining the modulating effect of the test compounds on the proteolytic activity of the separase.

It was an object of the invention to gain further insight into the mechanism of sister chromatid separation, in particular to elucidate the mechanism by which separase exerts its proteolytic activity, which has been shown to be involved in this process. The mechanism of separase activation provides the basis for developing improved enzymatic assays to identify modulators, in particular inhibitors of separase, in order to provide drugs that exert their effect by modulating, in particular inhibiting, sister chromatid segregation.

The identification of small molecule inhibitors of separase requires enzyme assays in which the protease activity of separase can be directly measured. Preferably, such assays are adaptable to high throughput formats so that large libraries of chemical compounds can be tested for their ability to inhibit separase. The previously reported experiments demonstrating that separase is associated with a protease activity were performed with separase isolated in small scale by immunoprecipitation or affinity chromatography from either yeast or human cells (Uhlmann et al., 2000; Waizenegger et al., 2000). Along these lines, WO00/48627 suggests to use full-length separase, preferably in recombinant form, for performing protease assays.

The protease activity of separase is tightly regulated during the cell cycle, ensuring that the ability of separase to cleave cohesin and thereby to dissolve sister chromatid cohesion is not activated before the transition from metaphase to anaphase. Work in budding yeast and human cells has shown that prior to anaphase separase is inhibited by a protein called securin (Ciosk et al., 1998; Uhlmann et al., 1999; Waizenegger et al., 2000). Securin binds to separase until securin is ubiquitinated by the anaphase-promoting complex and subsequently degraded by the 26S proteasome shortly before the onset of anaphase. The destruction of securin is thought to activate separase. In human cells, separase itself is cleaved at the same time as securin is destroyed, resulting in at least two C-terminal cleavage products called p55 and p60 (Waizenegger et al., 2000). These results suggest that the activity of separase is controlled by both securin destruction and separase cleavage. It is not known however and cannot be concluded from these results, which form of the separase, i.e. the full-length form or the cleavage products of separase, represent the active form of the protease or whether and in which way securin has a potential to contribute to the activation of separase.

To establish a separase protease assay suitable for high throughput format it is essential to know which form of separase represents the active enzyme and how the active form of separase can be obtained.

The present invention provides the first evidence that the cleaved forms of separase represent the active protease and that N- and C-terminal cleavage products of separase remain physically associated. The present invention further provides evidence that securin inhibits separase by directly binding to it. Securin binding to separase could either directly block the access of substrates to the active site of separase or it could keep separase in a conformation in which its active site is not accessible to substrates. The present invention further provides evidence that it is the autocatalytic reaction, i.e. the reaction in which separase cleaves itself, which is responsible for the cleavage of separase into its active form. Finally, the present invention shows that active forms of tagged recombinant full-length separase can be obtained and that fluorogenic peptide substrates are useful to measure the protease activity of separase.

It was previously reported that active human separase can be obtained by immunoprecipitating separase from human cell extracts (Waizenegger et al., 2000). Briefly, antibodies specific for the C-terminus of separase coupled to beads are incubated in lysates obtained from human cultured cells (for example of the line HeLa) which have previously been arrested in mitosis by treatment with microtubule poisons such as nocodazole. After removal of the cell lysate the antibody beads with bound separase are incubated in mitotic Xenopus laevis egg extracts which are able to ubiquitinate and degrade the securin protein which is bound to separase when it is immunoprecipitated from mitotic human cell lysates. During the incubation in mitotic Xenopus extracts the majority of securin is degraded, but in addition a major portion of human separase is also cleaved. After washing away the Xenopus egg extracts the antibody beads with bound separase are incubated with either purified cohesin complexes or with recombinant SCC1, which is the subunit of the cohesin complex that is cleaved by separase. The activity of separase is then monitored by analyzing the cleavage of SCC1, which can be analyzed by SDS gel electrophoresis and subsequent immunoblotting with antibodies to SCC1. If recombinant radioactively labeled SCC1 is used the gels can also be analyzed by autoradiography or Phosphorimaging.

In the experiments of the invention (Example 1), it was found that securin is able to bind to separase. It was shown that securin can bind to the cleaved form of separase as well as to the full-length separase. It was found that securin binding to separase inhibits the protease activity of separase (FIG. 1).

To further study the mechanism of separase activation it was tested if two different peptide inhibitors developed to inhibit separase from budding yeast are able to inhibit the protease activity of human separase. It was found that the concentration of these peptide derivatives required to inhibit human separase was similar to the concentration needed to inhibit separase from budding yeast (compare FIG. 2, upper panel and WO 00/48627, Uhlmann et al., 2000). It was further observed that the formation of the p55 cleavage product of human separase was largely inhibited by both peptides at the same concentration at which the ability of separase to cleave SCC1 was inhibited The obtained results suggest that separase activity itself is required for separase cleavage, i.e. that separase cleavage occurs autocatalytically.

Peptide inhibitors developed on the basis of the cleavage recognition site of human SCC1, “DREIMR”, were also able to inhibit separase at a similar concentration as the above mentioned peptide inhibitors (FIG. 3).

It was further tested which form of separase represents the active protease. It was found that the peptide inhibitor Bio-SVEQGR-amk bound exclusively to the cleaved forms of human separase when the peptide derivative was added to separase after its activation in mitotic Xenopus extracts. This observation shows that the active site of separase is accessible to Bio-SVEQGR-amk in the cleaved, but not in the residual amount of full-length separase to which securin is still bound (FIG. 4). These results suggest that the cleaved forms of separase represent active forms of the protease.

The obtained results also suggest that the full-length form of separase is also transiently active, presumably once its bound inhibitor securin has been destroyed, but that this form is normally labile because it is further processed into the cleaved forms by autocleavage.

It was shown that securin inhibits separase by directly or indirectly blocking the access of substrates to the active site of separase (FIG. 4D). It was further shown directly that separase cleavage can occur autocatalytically in trans and two cleavage sites in human separase were mapped (FIG. 8).

To test if the N- and C-terminal fragments of separase remain non-covalently associated after cleavage, tagged forms of separase were generated. During the course of these experiments it was recognized that recombinant separase expressed from the published cDNA sequence (KIAA 0165) was not the full length human enzyme but instead a fragment lacking the N-terminus of separase. The 5′-end of the human separase cDNA that encodes the missing N-terminus was therefore cloned. Subsequently, N-terminally FLAG-tagged full-length separase was transiently expressed in HeLa cells and isolated by immunoprecipitation with an anti-FLAG antibody. The immunoprecipitates were incubated in mitotic Xenopus egg extracts to allow securin destruction and separase cleavage. The finding that both C-and N-terminal separase fragments could be detected in the immunoprecipitates, suggested that the N- and C-terminal separase fragments remain associated after cleavage (FIG. 6).

Together, the obtained results suggest the model for the activation of human separase that is shown in FIG. 7. According to this model, full-length separase (p200) is associated with securin and represent the inactive state of the protease. Upon proteolysis of securin a labile full-length form of separase exists. The cleaved forms of separase stay physically associated with the N-terminal fragment(s) and represent the stabile, active enzyme.

The findings of the present invention show that protease assays for identifying separase inhibitors can be based on an active form of human separase, which is present in the cleaved forms of separase. These forms have the advantage to be more stable than the complete separase molecule and are thus expected to be better suitable for being employed in a high throughput format assay.

In order to obtain one or more active, stable forms of separase, the following steps can be taken:

Recombinant forms of the p55 and p60 cleavage products (Waizenegger et al., 2000), or potential other active cleavage products of separase, are produced in suitable expression systems, purified and tested for their ability to cleave SCC1, or a fragment thereof that contains the separase cleavage site, in vitro. Standard expression systems such as E. coli, budding yeast, Baculovirus infected Sf9 and Hi5 insect cells and transfected mammalian, e.g. human cells can be used. For purification, standard biochemical protocols can be used, e.g. those described in WO00/48627 for obtaining separase.

If p55 or p60 (or another, natural or synthetic, C-terminal fragment of human separase) alone or in combination with another fragment is sufficient for separase activity, the respective fragment (or a combination of fragments) can be employed in the protease assay (as described in WO00/48627) as a substitute for the full-length separase molecule.

Since the N- and C-terminal cleavage products of separase remain associated with each other (see above, FIG. 6) it is also possible that both the N- and the corresponding C-terminal fragments will be required to obtain active recombinant separase. The two or more fragments can either be expressed individually, purified and then mixed together, or they can be co-expressed in expression systems as listed above, and the obtained complexes containing both N- and C-terminal fragments are purified. All of these forms, e.g. all combinations of C-terminal and corresponding N-terminal fragments, will then be tested for their ability to cleave SCC1 in vitro. If any of the complexes described above yields human separase activity, the respective complex of separase fragments can be employed in the proteolytic assay in the same manner as a C-terminal fragment by itself, as described above.

If the above-described approach using complexes comprising various separase fragments does not exhibit human separase activity, full-length human separase is expressed in expression systems as listed above, the recombinant protein is isolated and activated, e.g. by incubation in mitotic Xenopus egg extracts, to induce its activation by cleavage.

In parallel, complexes of separase and securin can be generated to investigate whether the binding of securin to separase may not only inhibit separase but may also be required for its subsequent activation. To test this possibility, either recombinant securin is added to recombinant separase after their individual expression and purification, or securin and separase are co-expressed in expression systems as above. All forms of separase, i. e. full-length separase, separase fragments or combinations of fragments, with and without transiently bound securin, are tested for their ability to cleave SCC1 in vitro after incubation of the different forms of separase in mitotic Xenopus extracts.

In the case that these experiments result in the finding that securin is required for human separase activity, the assay is performed by employing the respective form of separase (fragment(s)) in combination with securin. The separase (fragment) is activated in the presence of securin in cell extracts, e.g. Xenopus laevis cell extracts. Preferably, as for the other assay components, securin, or a fragment thereof that proves to be sufficient for activation of separase (if not stated otherwise, “securin” also stands for an active fragment thereof) is employed in recombinant form, based on the cDNA sequence (Lee et al., 1999; Zhang et al, 1999). In order to obtain a protein complex for the assay, the separase (fragment) and securin can either be expressed and purified separately and then combined or they can be co-expressed and co-purified; as described above.

The results of FIG. 6 shows that the latter approach is feasible. Flag-tagged separase was coexpressed with myc-tagged securin in HeLa cells. Coexpression with securin lead to a higher yield of separase. Mitotically actived separase immunoprecipitates were able to cleave SCC1.

Once the active form(s) of human separase have been obtained by one or more of the methods described above, synthetic peptide substrates for separase are designed and synthesized that allow the simple detection of protease activity in high throughput format, e.g. by fluorogenic methods. The proteolytic assays suitable for this purpose have been described in WO00/48627. By way of example, substrate peptides containing the separase recognition sequence (see WO00/48627) that carry a C-terminal fluorophore such as a 7-amino-4-methyl-coumarin group (AMC) are synthesized by standard methods. The cleavage of AMC (or other fluorophore groups used) results in a rise in fluorescence which can be measured fluorometrically. In an experiment of the present invention, the activity of mitotically activated immunoprecipitates of separase (Waizenegger et al., 2000) was measured fluorometrically by using AMC-coupled peptides based on the cleavage recognition sites of human separase, “SFEILR”. The result of this experiment provides the basis for the development of a screening assay for identifying separase inhibitors. For conducting this assay in the high throughput mode, compounds, e.g. from chemical or natural product libraries, can be tested for their ability to inhibit the cleavage of fluorogenic peptide substrates by the active form(s) of human separase, which is preferably employed in the screen in recombinant form.

Similarly, the approaches as described above can be used to determine the active forms of separase from other eukaryotic organisms, to generate these forms as recombinant active proteins and to establish screening method for identifying inhibitors of these enzymes.

The present invention relates to method for identifying a compound that has the ability of modulating sister chromatid separation by inhibiting the proteolytic activity of separase, characterized in that an active separase in the form of

a) one or more separase fragment(s), optionally upon activation in the presence of securin, or

b) full-length separase upon activation in the presence of securin,

is incubated in the presence of a separase substrate, with a test compound and that the modulating effect of the test compounds on the proteolytic activity of the separase is determined.

Any variation of the proteolytic screening assay method of the invention, e.g. carried out with one or more separase fragments, in the presence or absence of securin, can be carried out according to standard methods, in particular as described in WO 00/48627:

Various assay methods for identifying protease inhibitors that are useful in the present invention and are amenable to automation in a high-throughput format have been described, e.g. the radiometric method described by Cerretani et al., 1999, for hepatitis C virus NS3 protease, the method based on fluorescence quenching described by Ambrose et al., 1998, or by Taliani et al., 1996, the microtiter colorimetric assay fot the HIV-1 protease described by Stebbins and Debouck, 1997, the fluorescence polarization assay described by Levine et al., 1997 (reviewed by Jolley, 1996), the method using immobilized peptide substrates described by Singh et al., 1996, the assay used for studying the inhibition of cathepsin G, using biotinylated and cysteine-modified peptides described by Brown et al., 1994. A further example for a suitable assay is based on the phenomenonon of fluorescence resonance energy transfer (FRET), as described by Gershkovich et al., 1996 or by Matayoshi et al., 1990. Additional examples for assays that may be used in the present invention for a high-throughput screening method to identify inhibitors of separase activity were described by Gray et al., 1994, Murray et al., 1993, Sarubbi et al., 1991.

Fluorescent or radioactive labels and the other reagents for carrying out the enzymatic reaction on a high-throughput scale are commercially available and can be employed according to supplier's instructions (e.g. Molecular Probes, Wallac). The specific assay design depends on various parameters, e.g. on the size of the substrate used. In the case of using a short peptide, the fluorescence quenching or the fluorescence resonance energy transfer methods are preferred examples for suitable assay technologies.

The fluorescence quenching (Resonance Energy Transfer _{”}RET“) assay relies on synthetic substrates which are capable of direct, continuous signal generation that is proportional to the extent of substrate hydrolysis. The substrate peptide carries a fluorescent donor near one end and an acceptor near the other end. The fluorescence of the substrate is initially quenched by intramolecular RET between donor and acceptor. Upon cleavage of the substrate by the protease the cleavage products are released from RET quenching and the a fluorescence proportional to the amount of cleaved substrate can be detected. In Example 9, this type of assay is exemplified by use of AMC; which serves as a donor fluorophore and in the case of the separase-specific peptide substrates the amino acid bonds of the peptides function as acceptor chromophores.

An assay of this type may be also carried out as follows: the solution of the labeled substrate (e.g. the peptide labeled with 4-[[4′(dimethylamino)phenyl]azo]benzoic acid (DABCYL) at the one end and with 5-[(2′-aminoethyl)amino]naphtalenesulfonic acid (EDANS) at the other end or labeled with benzyloxycarbonyl at the one end and with 4-aminomethylcoumarin at the other end) in assay buffer is pipetted into each well of black 96-well microtiter plates. After addition of the test substances in the defined concentration, the separase activity containing solution is added to the wells. After incubation under conditions and for a period of time sufficient for the proteolytic cleavage reaction, e.g. for 1 hour at room temperature, the fluorescence is measured in a fluorometer at the excitation wavelength, e.g. at 340 nm, and at the emission wavelength, e.g. at 485 nm.

In the case of using the FRET assay, labeling pairs that are suitable for the method of the invention are commercially availabe, e.g. Europium (Eu) and Allophycocyanin (APC), Eu and Cy5, Eu and PE (Wallac, Turku, Finland).

The compounds identified in the above methods have the ability to interfere with sister chromatid separation by modulating the proteolytic activity of separase.

The present invention also relates to compounds which act as inhibitors of separase for use in human therapy, in particular cancer therapy.

In a further aspect, the invention relates to a pharmaceutical composition which contains, as the active ingredient, one or more compounds which interfere with or modulate sister chromatid separation by inhibiting separase activity.

In a preferred embodiment, the invention comprises pharmaceutically active compounds and their use in therapy, which are small chemical molecules that have been identified as separase inhibitors in the screening method of the invention.

As an alternative to identifying small molecules in a screening method, separase inhibitors can be obtained starting from the recombinant active separase. In this approach, synthetic peptide derivatives, exemplified by derivates of SVEQGR, DREIMR, SFEILR or EWELLR (e.g. Bio-SVEQGR-amk) can be used as the structural basis to develop peptidomimetic molecules that inhibit separase. For inhibitors of human separase, the cleavage sequence of human SCC1 or human separase can preferably be used. The assays described above using recombinant active separase and peptide substrates, e.g. fluorogenic peptides, can be used to optimize such compounds.

Inhibitors of human separase activity identified in the screening methods of the invention or based on rational inhibitor design can be used as cytotoxic therapeutics for the treatment of diseases that are caused by uncontrolled cell proliferation, such as cancers, leukaemias, or cardiac restenosis. Species specific inhibitors of separase from eukaryotic pathogenic microorganisms can be used to treat infectious diseases caused by such microorganisms, for example infections caused by pathogenic fungi or diseases caused by parasites such as Leishmania species.

To address how separase inhibition affects cell cycle progression, RNA interference experiments can be used to knock out separase expression in human cultured cells, according to known methods, as described, e.g. by Elbashir et al, 2001.

Influencing the process of sister chromatid separation may be also beneficial in preventing birth defects caused by missegration of chromosomes in human meioses. For example, since cases of human aneuploidy such as Down's syndrome may be caused by premature separation of sister chromatids (Griffin, 1996), the use of a drug that inhibits separase activity might be able to reduce precocious sister separation and thereby the incidence of aneuploidy in human fetuses.

Thus, in a further aspect, the invention relates to separase inhibitors for the prevention of birth defects caused by missegration of chromosomes in human meioses.

The efficacy of compounds identified as separase inhibitors in the method of the invention, can be tested for in vivo efficacy either on yeast cells or in mammalian cells. Effective compounds should block (or at least in some way interfere with) sister chromatid separation, which can be measured, e.g. by using CenV-GFP in yeast, as described by Ciosk et al., 1998, or standard cytological techniques in mammalian cells. Compounds effective in tumor therapy should be either cytostatic or cytotoxic. Substances whose potential for therapeutic use has been confirmed in such secondary screens can be further tested for their effect on tumor cells.

To test the inhibition of tumor cell proliferation, primary human tumor cells are incubated with the compound identified in the screen and the inhibition of tumor cell proliferation is tested by conventional methods, e.g. bromo-desoxy-uridine or ³H incorporation. Compounds that exhibit an anti-proliferative effect in these assays may be further tested in tumor animal models and used for the therapy of tumors.

Toxicity and therapeutic efficacy of the compounds identified as drug candidates by the method of the invention can be determined by standard pharmaceutical procedures, which include conducting cell culture and animal experiments to determine the IC₅₀, LD₅₀, the ED₅₀. The data obtained are used for determining the human dose range, which will also depend on the dosage form (tablets, capsules, aerosol sprays, ampules, etc.) and the administration route (oral, buccal, nasal, paterental or rectal). A pharmaceutical composition containing the compound as the active ingredient can be formulated in conventional manner using one or more physologically active carriers and excipients. Methods for making such formulations can be found in manuals, e.g. “Remington Pharmaceutical Sciences”.

Separase inhibitors may also be useful in applications which aim at the deliberate polyploidisation of plant cells for crop development. In yeast, it has been shown that inhibition of separase activity prevents chromosome separation without blocking cell cycle progression and therefore gives rise to cells with increased ploidy. Inhibitors that block separase's protease activity could therefore be used to increase the ploidy of any eukaryotic cell, including all plant cells. Increasing the ploidy of plant cells is useful for 1) producing larger plants, 2) for increasing the ploidy of breeding stocks, and 3) for generating fertile hybrids.

Therefore, the present invention relates, in a further aspect, to separase inhibitors for the treatment of plant cells for increasing their ploidy.

To identify separase inhibitors that are useful for the above-mentioned agricultural purposes, the screening method of the invention can be easily adapted by employing plant components, i.e. a plant separase and a plant homolog of SCC1. Sequence homologs of plant separase and SCC1 are present in databases, e.g. of the Arabidopsis thaliana genome.

Separase inhibitors which impair sister chromatid separation may also be used in cytological analyses of chromosomes, for example, in medical diagnoses of chromosome structure.

### BRIEF DESCRIPTION OF THE FIGURES

FIG. 1: Securin acts as an inhibitor for separase

FIG. 2: Yeast peptides inhibit proteolytic activity of human separase in similar concentration as they inhibit yeast separase and influence the processing of human separase

FIG. 3: Human peptides inhibit proteolytic activity of human separase in a similar concentration as the yeast peptides do

FIG. 4: Addition of yeast peptides at different stages during the activation of separase suggests that the cleaved forms are the active forms of separase and securin binding blocks access of peptide substrates to the active site of separase

FIG. 5: Mitotically activated separase has autocatalytic activity

FIG. 6: N- and C-terminal cleavage products of human separase stay associated after mitotic cleavage and ectopically expressed separase is able to cleave human SCC1

FIG. 7: Working model for the activation of human separase

FIG. 8: Mapping of the cleavage sites in human separase

FIG. 9A: Principle of fluorogenic separase inhibitor screening assay

B: Processing of a separase peptide substrate by trypsin

FIG. 10: In vitro assay using activated separase bound to microbeads

FIG. 11: Transcleavage separase screening assay showing inhibiting effect of small molecular compounds

### MATERIAL AND METHODS

SCC1 in Vitro Cleavage Assay

Inactive human separase (separase) immunoprecipitates were activated in mitotic Xenopus egg extracts (for details see Waizenegger et al., 2000). 30 microliter beads were incubated with 40 μg bacterially expressed wildtype securin, destruction box deleted form of securin (Gmachl et al., 2000) or BSA diluted in XB+1 mM DTT for 40 min at RT. Subsequently the beads were washed with XB+1 mM DTT and with TBS+0.5 M NaCl+1 mM DTT+0.5% TWEEN20, followed by one wash with XB+1 mM DTT. To control the rebinding of securin an aliquot of 5 μl was taken per assay and subsequently analysed by immunoblotting with antibodies against separase and securin.

The beads were used for the SCC1 in vitro cleavage assay: 20 μl beads were mixed with 30 μl of the following SCC1 in vitro translation mix (SCC1 myc IVT+1 μl PLK-GST+0.3 μl M MgCl₂, 0.3 μl 100 mM ATP, 0.12 μl 250 mM EGTA, 13.78 μl XB+1 mM DTT) and incubated at 22° C. and 1200 rpm. 5 μl were taken per time point, the reaction was stopped by addition of SDS loading buffer. Samples were analyzed by immunoblotting with mouse monoclonal antibodies against myc (9E10).

### Modifications:

A. Mitotically activated separase immunoprecipitates were preincubated with yeast peptides (Uhlmann et al., 2000) before they were used in the SCC1-cleavage assay. Biotin-SVEQGR-amk or Biotin-SVEQGR-cmk were used at different concentration (0.1, 1, 10, 100, 1000 μM in XB+0.5 mM DTT). Separase immunoprecipitates were incubated for 10 minutes, 22° C. and 1200 rpm. The SCC1 in vitro cleavage assay was performed after washing once with XB+0.5 mM DTT (see above). Human peptides, Biotin-DREIMR-amk or DREIMR-amk, were diluted and used as above described for the yeast peptides.

B. Separase immunoprecipitates were incubated with 100 μM Biotin-SVEQGR-amk or with DMSO prior mitotic activation. Incubation for 10 minutes, 22° C. and 1000 rpm. After washing twice in XB+1 mM DTT the activation was performed in mitotic Xenopus egg extracts.

Interphase Xenopus egg extracts were driven into mitosis in the presence of 1 mM Biotin-SVEQGR-amk or DMSO. Those mitotic Xenopus egg extracts were used to activate a batch of separase immunoprecipitates.

Biotin labeled peptides were detected via immunoblotting according to Faleiro et al., 1997.

C. Mitotically activated separase immunoprecipitates were preincubated with either 8 μg recombinant securin or with 8 μg of a C-terminal truncated version of cyclinB in 100 μl XB+1 mM DTT for 30 min at 22° C. and 1250 rpm. Subsequently the beads were washed twice with TBS+0.5 M NaCl+1 mM DTT+0.5% Tween 20 and twice with XB+1 mM DTT. Thereafter beads were incubated with 100 μl of 100 μM Bio-DREIMR-amk in XB+1 nM DTT. After 10 min incubation at 22° C. and 1250 rpm beads were again washed and analyzed by immunoblotting.

D. A separase construct tagged with one myc epitope at its C-terminus was in vitro transcribed and translated and then used as a substrate for separase bound to beads. The reaction mix was supplemented as described above for the SCC1 cleavage assay. The myc tag allowed the discrimination between separase as a substrate and as an enzyme.

### EXAMPLE 1

Securin Acts as an Inhibitor for Separase

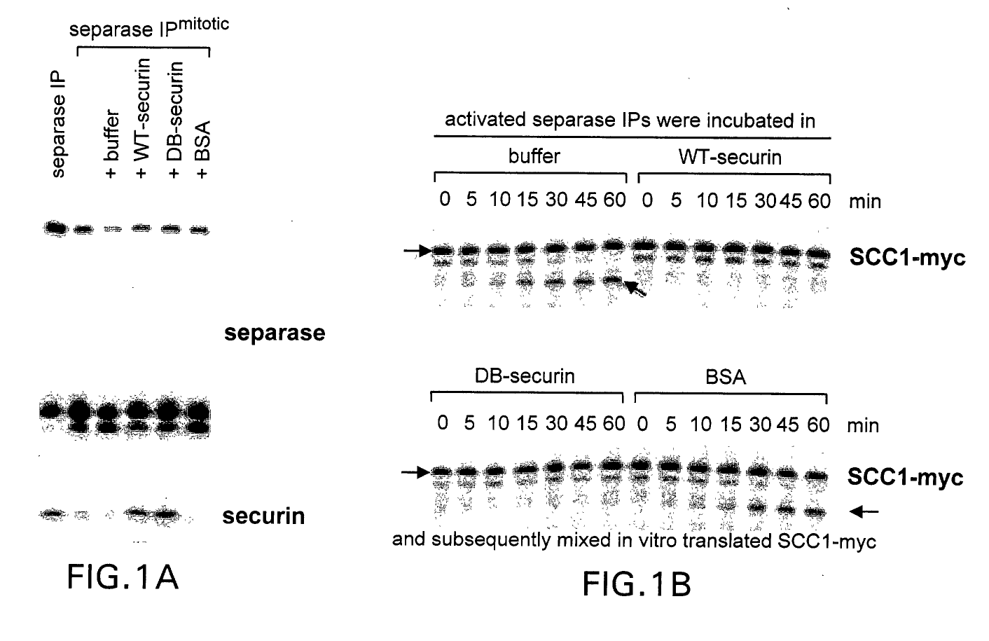

To test if securin inhibits separase by directly binding to it and if it is still able to bind and inhibit the cleaved form of separase human securin was expressed in E. coli and purified. When the recombinant securin was added to separase that had been isolated by immunoprecipitation and had been activated in mitotic Xenopus extracts as above it was observed that securin was able to bind to separase (FIG. 1A). Importantly, the amount of securin that bound to separase was at least as high as the amount of securin that had been bound to separase originally before securin was degraded by incubating the separase immunoprecipitates in mitotic Xenopus extracts, although the majority of separase had been cleaved during the incubation in the Xenopus extract. This result shows that securin can bind to the cleaved form of separase as well as to the full-length separase.

When the cleaved form of separase to which recombinant securin had been bound was incubated with recombinant SCC1, no cleavage of SCC1 was detected (FIG. 1B), demonstrating that recombinant securin is able to inhibit the protease activity of separase by directly binding to it. Because the majority of separase used in this experiment was present in its cleaved form these observations suggest that it is the cleaved form of separase that is the active protease which cleaves SCC1, unless it is inhibited by the rebinding of securin. Identical results were obtained with two different forms of securin, the wildtype protein (WT-securin) and a form that can not be destroyed by ubiquitin-dependent proteolysis (DB-securin; FIGS. 1A and B).

FIG. 1A: Separase immunoprecipitates (separase IP) obtained from Nocodazole arrested HeLa cells and bound by antibodies against the C-terminus of separase coupled to beads were activated in mitotic Xenopus egg extracts (separase IP^{mitotic}). Mitotically activated separase immunoprecipitates were either incubated in buffer, wildtype securin, destruction box deleted securin or in BSA. Aliquots were taken and analysed by immunoblotting with antibodies against separase and securin.

FIG. 1B: Activated separase immunprecipitates which were either incubated with buffer, wildtype securin, destruction box deleted securin or BSA were incubated SCC1-myc reaction mix. Aliquots were taken at indicated timepoints and analysed by immunoblotting with antibodies against myc. Cleaved SCC1 is marked by arrows.

### EXAMPLE 2

Yeast Peptides Inhibit Proteolytic Activity of Human Separase in Similar Concentration as They Inhibit Yeast Separase and Influence the Processing of Human Separase

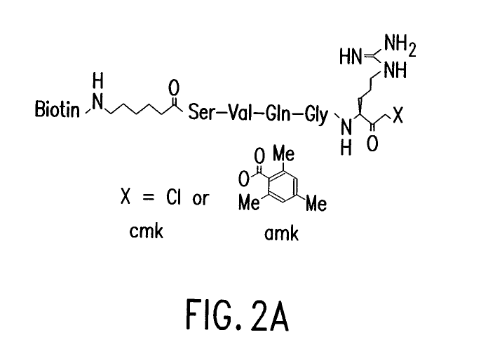

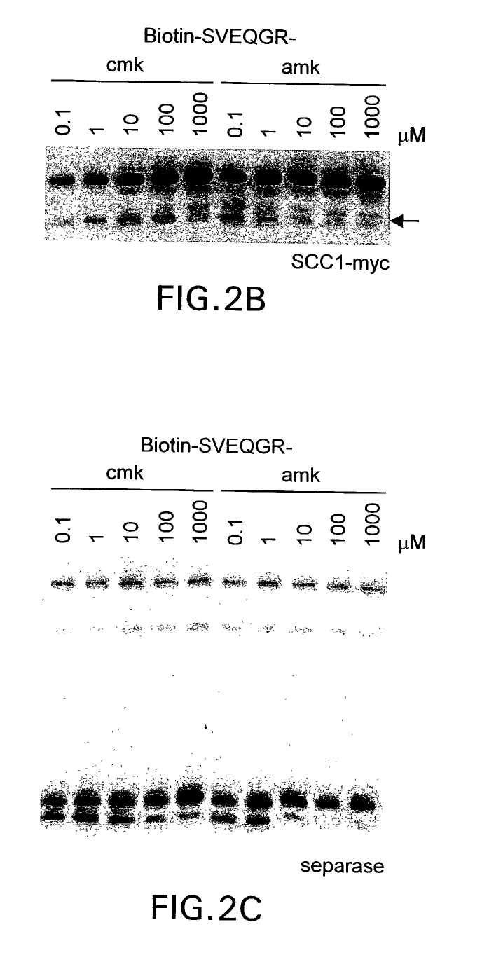

To further study the mechanism of separase activation it was tested if two different peptide inhibitors developed to inhibit separase from budding yeast (Uhlmann et al., 2000) are able to inhibit the protease activity of human separase. These inhibitors are synthetic peptides containing the cleavage site of budding yeast SCC1, “SVEQGR”, where the last arginine residue represents the P1 site after which separase cleaves. The C-terminus of this peptide is either modified to a chloromethyl ketone (cmk) or to an acyloxymethyl ketone (amk). Both peptide derivatives are coupled to biotin moieties at their N-termini. The two inhibitors are therefore called Bio-SVEQGR-cmk and Bio-SVEQGR-amk. When these inhibitors were added to human separase that had been isolated by immunoprecipitation and had been activated in mitotic Xenopus extracts as above, it was observed that both inhibitors are able to block the ability of separase to cleave SCC1 (FIG. 2A). The concentration of these peptide derivatives required to inhibit human separase was similar to the concentration needed to inhibit separase from budding yeast (compare FIG. 2, upper panel and WO 00/48627, Uhlmann et al., 2000). It was further observed in this experiment that the formation of the p55 cleavage product of human separase was largely inhibited by both peptides at the same concentration at which the ability of separase to cleave SCC1 was inhibited (FIG. 2B).

FIG. 2A: The structure of the yeast peptides

FIG. 2B: Separase immunoprecipitates obtained from nocodazole arrested HeLa cells bound by antibodies against the C-terminus of separase coupled to beads were activated in mitotic Xenopus egg extracts. Subsequently samples were incubated with indicated concentrations of yeast peptides (Biotin-SVEQGR-cmk or Biotin-SVEQGR-amk). After a short wash samples were mixed with SCC1-myc reaction mix for 1 hour. Samples were analysed by immunoblotting with antibodies against myc. Cleaved SCC1 is marked by an arrow.

FIG. 2C: The samples (see B) were immunblotted with antibodies against separase.

### EXAMPLE 3

Human Peptides Inhibit Proteolytic Activity of Human Separase in a Similar Concentration as the Yeast Peptides Inhibit Yeast and Human Separase

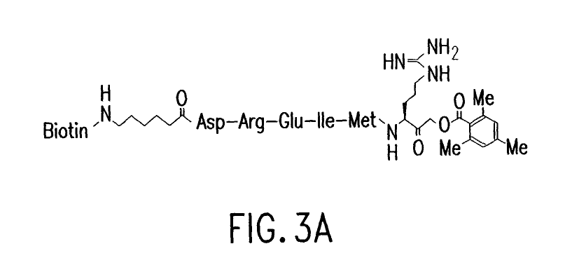

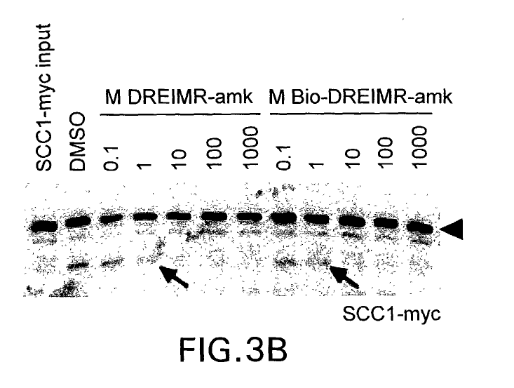

Separase immunoprecipitates obtained from nocodazole-arrested HeLa cells bound by antibodies against the C-terminus of separase coupled to beads were activted in mitotic Xenopus egg extracts. Subsequently samples were incubated with the indicated concentrations of human peptides (DREIMR-amk or Biotin-DREIMR-amk). After washing samples were mixed with SCC1-myc reaction mix for 1 hour. Samples were analysed by immunoblotting with antibodies against myc. For control a sample was treated with the same concentration of DMSO which was used for the solubilization of the peptides (DMSO). The SCC1-myc reaction mix was loaded as an input control (SCC1-myc input). It was found that peptide inhibitors developed on the basis of the cleavage recognition site of human SCC1, “DREIMR”, were able to inhibit separase at a similar concentration as the peptide inhibitors derived from yeast (FIG. 3).

FIG. 3A shows the structure of the human peptide

FIG. 3B shows that human peptide derivatives inhibit the SCC1 cleavage activity of separase. Full length SCC1 is indicated by an arrowhead, cleaved SCC1 is marked by an arrow.

### EXAMPLE 4

Addition of Yeast Inhibiting Peptides at Different Stages During the Activation of Separase Suggests that the Cleaved Forms are the Active Forms of Separase and that Securin Blocks Access of Substrates to the Active Site of Separase

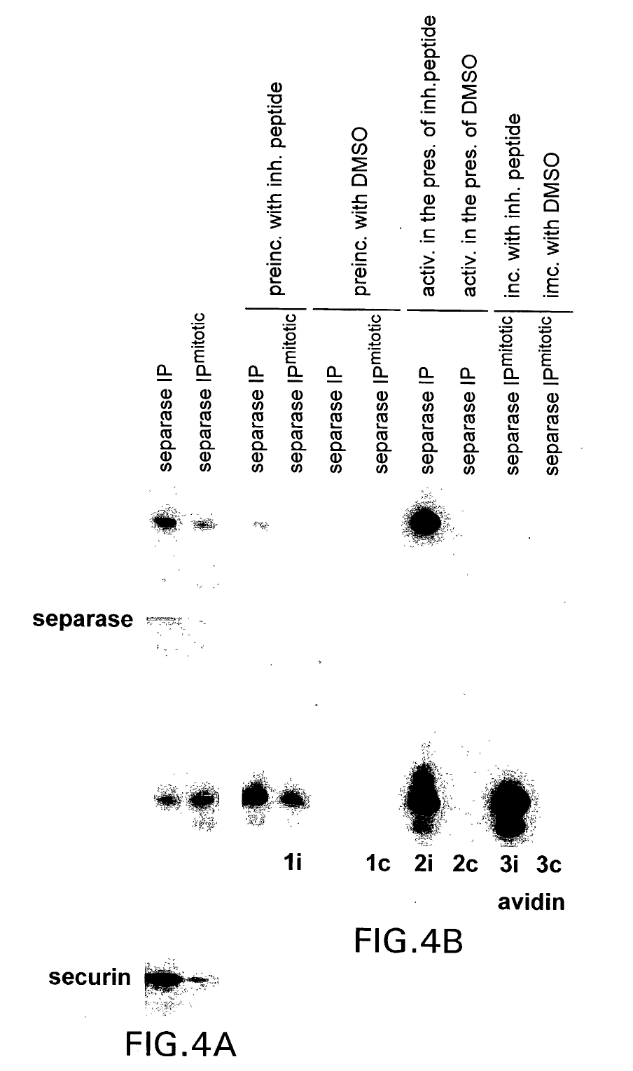

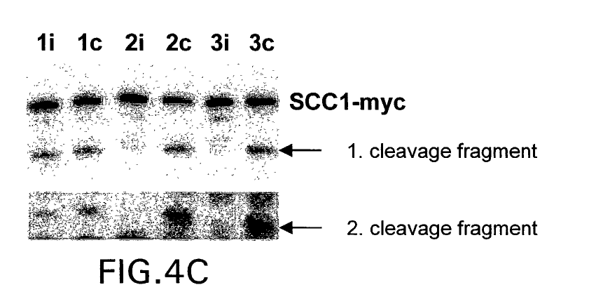

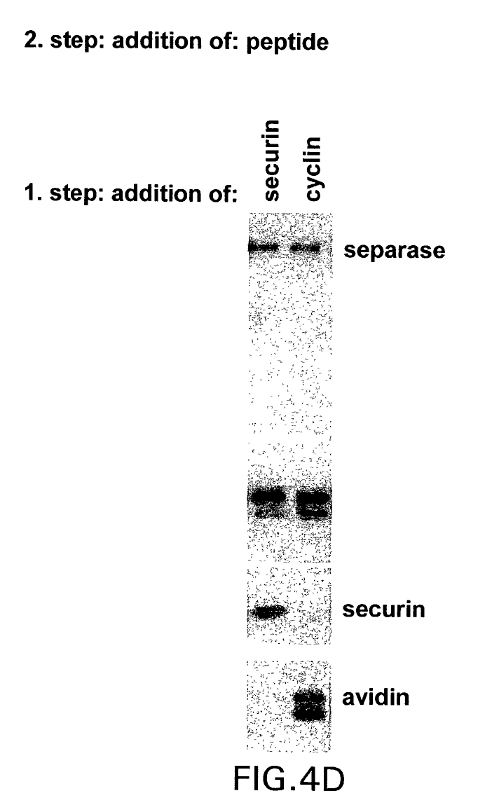

To further test which form of separase represents the active protease, the ability of Bio-SVEQGR-amk, the more effective one of the two yeast peptide inhibitors, to bind to different forms of separase, was tested. Previous work has shown that the peptide derivatives inhibit separase by covalently binding to an active site cysteine residue within separase (Uhlmann et al., 2000). These binding reactions can be directly visualized by separating the separase-inhibitor conjugate by SDS gel electrophoresis and by subsequently labeling the biotin moiety on the peptide derivative by streptavidin (Uhlmann et al., 2000). By using this method it was found that Bio-SVEQGR-amk bound exclusively to the cleaved forms of human separase when the peptide derivative was added to separase after its activation in mitotic Xenopus extracts (FIG. 4B, lane 3i). This treatment inhibited SCC1 cleavage by separase (FIG. 4C, lane 3i). This observation shows that the active site of separase is accessible to Bio-SVEQGR-amk in the cleaved, but not in the residual amount of full-length separase, confirming the conclusion from the securin addback experiments that the cleaved forms of separase represent active forms of the protease.

When Bio-SVEQGR-amk was added to human separase immunoprecipitates before separase had been activated in mitotic Xenopus extracts, only the small amount of p60 that is already present in these immunoprecipitates was labeled by the peptides, whereas full-length separase was not (FIG. 4B, lane 1i), further confirming that the active site of separase is only accessible in the cleaved forms. When the peptide inhibitor was washed away before the separase was subsequently incubated in mitotic Xenopus extracts separase could be activated normally to cleave SCC1 (FIG. 4C, lane 1i). This result suggests that the presence of securin prevents the binding of Bio-SVEQGR-amk to the active site of separase, implying that securin inhibits separase by directly or indirectly blocking the access of substrates to the active site of separase.

When the activation of human separase immunoprecipitates in mitotic Xenopus extracts was carried out in the presence of Bio-SVEQGR-amk both the cleaved forms of separase and full-length separase were covalently labeled with the peptide (FIG. 4B, lane 2i) and separase was unable to cleave SCC1 (FIG. 4C, lane 2i). This observation suggests that the full-length form of separase is also transiently active, presumably once its bound inhibitor securin has been destroyed, but that this form is normally labile because it is further processed into the cleaved forms by autocleavage. It was further shown that binding of recombinant securin to cleaved active separase prevented the binding of peptide inhibitors to the active site of separase (FIG. 4D). This result suggests that securin inhibits separase by either directly or indirectly blocking the access of substrates to the active site of separase.

FIG. 4A: Separase immunoprecipitates (separase IP) obtained from Nocodazole arrested HeLa cells bound by antibodies against the C-terminus of separase coupled to beads were activated in mitotic Xenopus egg extracts (separase IP^{mitotic}) Aliquots were analysed by immunoblotting with antibodies against separase and securin.

FIG. 4B: Separase IPs (see A) were either preincubated with Biotin-SVEQGR-amk (preinc. with inh. peptide) or with DMSO (preinc. with DMSO), subsequently washed and aliquots were taken. Thereafter they were incubated in mitotic Xenopus egg extracts and washed again. Samples were taken for analysis (1i, 1c). Separase IPs were activated either in mitotic Xenopus egg extracts which were driven into mitosis in the presence of Biotin-SVEQGR-amk or DMSO, subsequently washed and aliquots were taken for analysis (2i, 2c). Already mitotically activated separase IPs (see A) were incubated with Biotin-SVEQGR-amk or DMSO, thereafter washed and aliquots were taken for analysis (3i, 3c). All samples were analysed by immunoblotting with avidin.

FIG. 4C: Samples 1i, 1c, 2i, 2c, 3i and 3c (see B) were mixed with SCC1-myc reaction mix for 1 hour and analysed by immunoblotting with antibodies against myc. Arrows indicate the first and second SCC1 cleavage product.

FIG. 4D: Activated separase immunoprecipitates bound by antibodies against the C-terminus of separase to beads were first incubated with recombinant securin or, for control, with recombinant truncated cyclinB. After washing the precipitates were incubated with the human peptide inhibitor. Thereafter, the immunoprecipitates were again washed and analyzed by immunoblotting.

### EXAMPLE 5

a) Cloning of Full Length Human Separase

The missing N-terminal part of human separase was amplified from a HeLa cDNA library by polymerase chain reaction using the following primers:

5′primer: ^{5′}GGCCAATTGATATCATGAGGAGCTTCAAAAGAG^{3′} (SEQ ID NO: 3)

3′primer: ^{5′}CAACTGTCCACTAGTTGGGTCAGG^{3′} (SEQ ID NO: 4)

The resulting DNA fragment was inserted via EcoRV and Spel into the existing truncated form of human separase (KIAA 0165). The complete coding sequence of human separase is shown in SEQ ID NO: 1, the amino acid sequence is shown in SEQ ID NO: 2.

b) Preparation of Recombinant Human Separase

Human separase was N-terminally tagged with a Flag epitope and transiently transfected in HeLa cells either in a single transfection or in a cotransfection with human securin which was C-terminally tagged with a myc epitope. For control HeLa cells were also transiently transfected with securin-myc. After 24 hours transfection 330 nM nocodazole was added for 18 hours. Cells were harvested, washed with PBS and cell extracts were generated as described in Waizenegger et al. 2000. These cell extracts were used to immunoprecipitate exogenous Flag-separase with mouse anti-Flag antibodies bound to sepharose. These immunoprecipitates were then incubated in mitotic Xenopus egg extracts and reisolated. The immunoprecipitates and the mitotically activated immunoprecipitates were analysed by immunoblotting with mouse antibodies against separase (7A6), securin (mouse serum) and Flag (M2, Stratagene). The mitotically activated immunoprecipitates were analysed for their activity (see above: SCC1 in vitro cleavage assay).

### EXAMPLE 6

Mitotically Activated Separase has Autocatalytic Activity

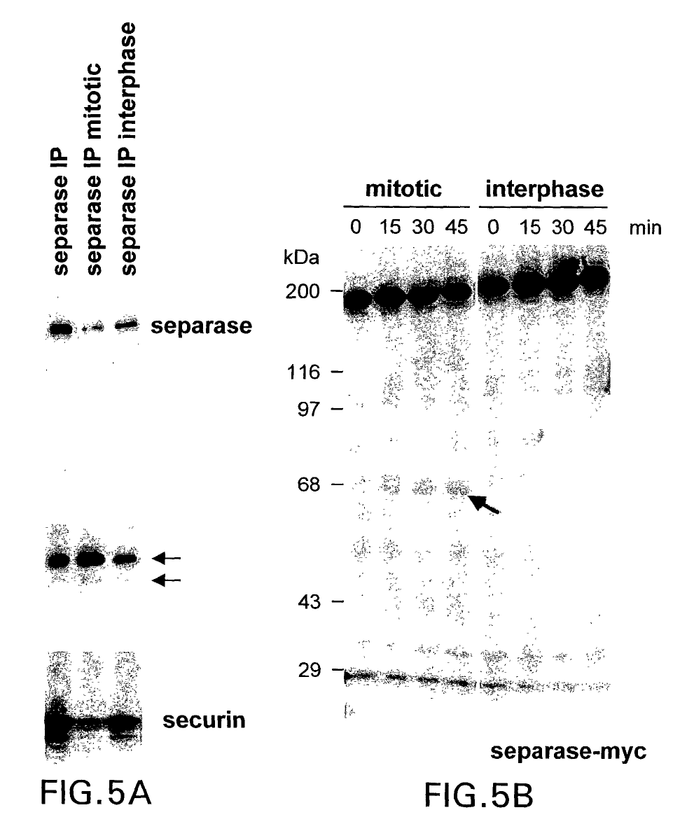

To test if separase has autocatalytic activity, a myc-tagged form of separase obtained by in vitro transcription and translation was used as a substrate. The transcription-translation reaction was carried out in the presence of ³⁵S-labeled methionine and cysteine, resulting in radiolabeled translation products. The in vitro translated separase was stable when incubated with separase immunoprecipitates which were incubated in interphase Xenopus egg extracts but in vitro translated separase was cleaved when it was incubated with active separase obtained by incubation of separase immunoprecipitates in mitotic Xenopus egg extracts (FIG. 5). These results show that separase cleavage can occur autocatalytically in trans. This allows to distinguish between separase acting as an enzyme and separase serving as a substrate.

FIG. 5 shows that only upon mitotic activation of separase separase-myc is autocatalytically cleaved.

FIG. 5A: Separase immunoprecipitates (separase IP) obtained from Nocodazole arrested HeLa cells bound by antibodies against the C-terminus of separase coupled to beads were either incubated in mitotic (separase IP^{mitotic}) or in interphase Xenopus egg extracts (separase Ip^{interphase}) Aliquots were analysed by immunoblotting with antibodies against separase and securin.

FIG. 5B: Separase immunoprecipitates were incubated either in a mitotic or interphase Xenopus egg extracts and then mixed with recombinant separase-myc reaction mix. At indicated time points samples were taken and analysed by immunoblotting with antibodies against myc.

### EXAMPLE 7

N-and C-terminally Cleavage Products of Separase Remain Physically Associated and Tagged Recombinant Human Separase is Active

To test if the N- and C-terminal fragments of separase remain non-covalently associated after cleavage, tagged forms of separase were generated.

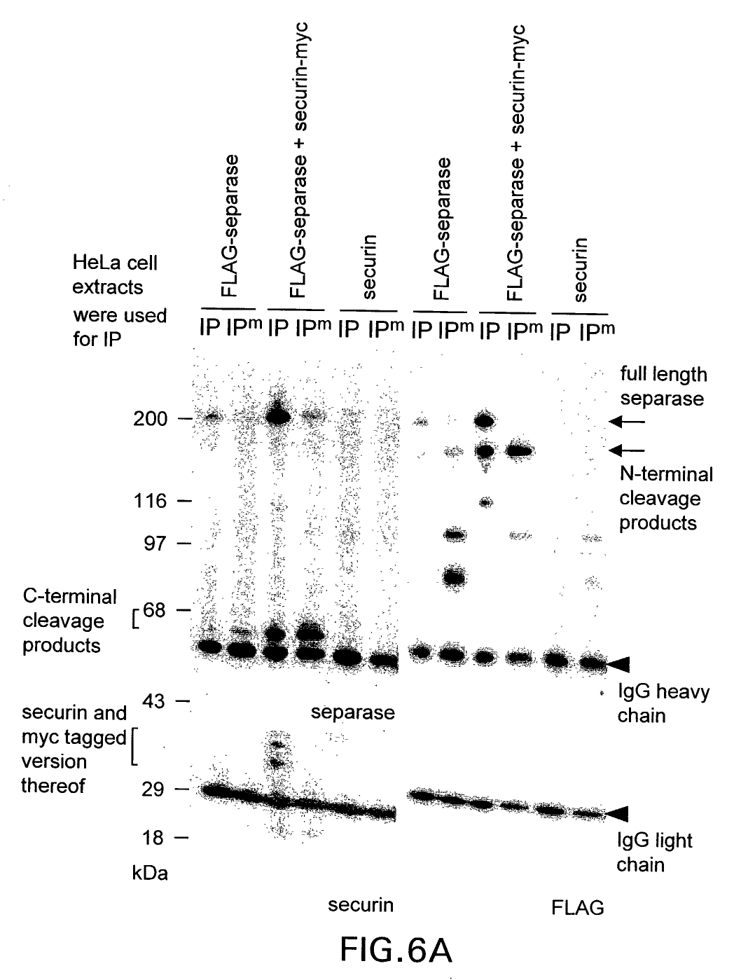

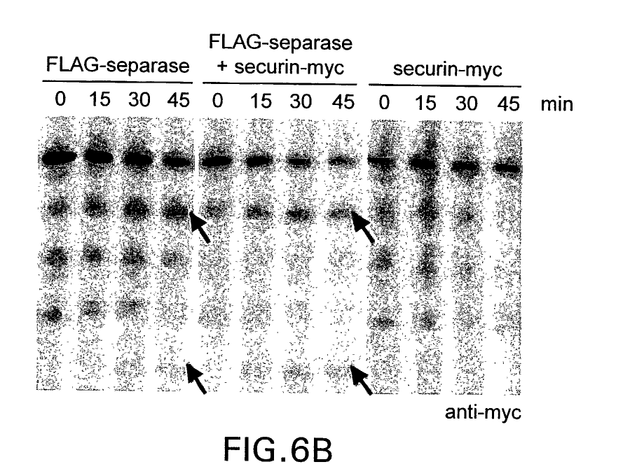

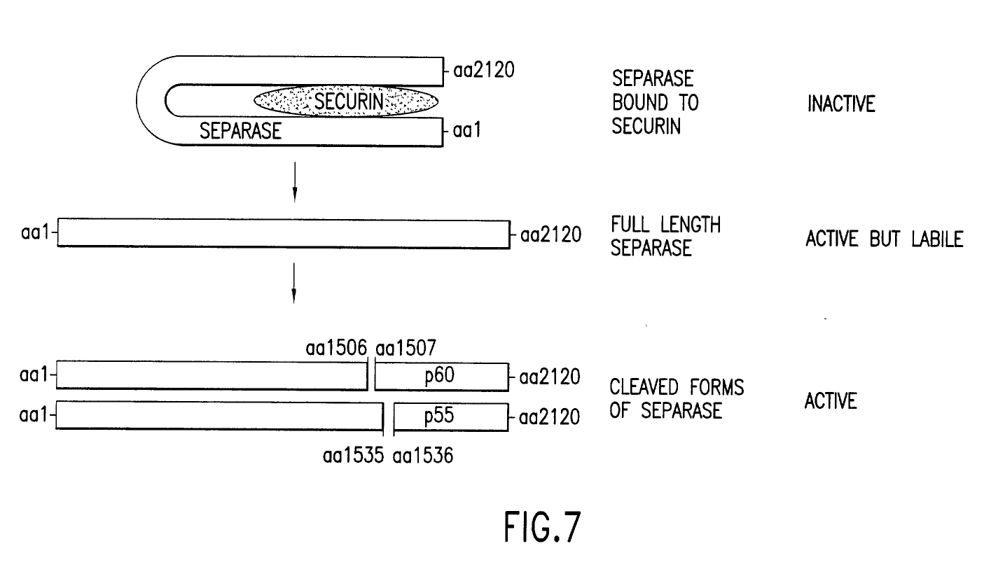

N-terminally FLAG-tagged full length separase was transiently expressed in HeLa cells as described in Example 5B and isolated by immunoprecipitation with an anti-FLAG antibody. The immunoprecipitates were incubated in mitotic Xenopus egg extracts to allow securin destruction and separase cleavage (FIG. 6A). After re-isolation the immunoprecipitates were immunoblotted with anti-separase antibodies, anti-securin antibodies or anti-FLAG antibodies. Both C-and N-terminal separase fragments could be detected in the immunoprecipitates, suggesting that the N- and C-terminal separase fragments remain associated after cleavage (FIG. 6A).

FIG. 6A: HeLa cells were transiently transfected with FLAG-separase, FLAG-separase and securin-myc or only with securin-myc. Mitotic extracts were performed from these cells and used for immunoprecipitation with anti-FLAG antibodies bound to sepharose (IP). The immunoprecipitates were activated in mitotic Xenopus egg extracts (IP^{m}). The immunoprecipitates were analysed by immunoblotting with antibodies against securin, separase (7A6) and FLAG.

FIG. 6B: Aliquots of above described immunoprecipitates were incubated with SCC1-myc reaction mix. At indicated time points samples were withdrawn and analysed by immunoblotting with antibodies against myc. Arrows indicate SCC1-myc cleavage products.

### EXAMPLE 8

Mapping of the Separase Recognition Sites

The cleavage sites in human separase were mapped by a method that has been previously used to map cleavage sites in SCC1 (WO00/48627). Briefly, truncated versions of the human separase cDNA were generated by polymerase chain reactions, and the resulting cDNAs were used directly for coupled in vitro transcription-translation reactions by using rabbit reticulocyte lysates. The transcription-translation reactions were carried out in the presence of ³⁵S-Iabeled methionine and cysteine, resulting in radiolabeled translation products. These were then separated by SDS gel electrophoresis side by side with the in vitro cleavage products of mitotically activated human separase immunoprecipitates which were detected by immunoblotting. The comparison of the electrophoretic mobility of a series of deletion mutants with the mobility of the in vitro cleavage products narrows down the regions of cleavage to about 10 amino acid residues. Because separase cleavage sites in all known organisms cleave after the sequence EXXR (where X represents any amino acid residue; WO 00/48627 ; Uhlmann et al., 1999; Buonomo et al., 2000; Hauf et al., 2001) it is assumed that SFEILR¹⁵⁰⁶ and EWELLR¹⁵³⁵ represent two of the separase cleavage sites.

N-terminally truncated separase cDNA was generated by polymerase chain reaction. The 5′primers contain a sequence with the T7 polymerase binding site

1. start aa 1487

^{5′}GAATTCTAATACGACTCACTATAGGATCCATGATCCCTGAGGAAGMC TGACTG^{3′} (SEQ ID NO: 5)

2. start aa 1507

^{5′}GAATTCTAATACGACTCACTATAGGATCCATGTCTGACGGGGAAGAC TCAGCCTC^{3′} (SEQ ID NO: 6)

3. start aa 1536

^{5′}GMTTCTMTACGACTCACTATAGGATCCATGGATTCCAGCAAGMGA AGCTGCCC^{3′} (SEQ ID NO: 7)

The following 3′primer was used:

^{5′}TTATTACCGCAGAGAGACAGGCMGCC^{3′} (SEQ ID NO: 8)

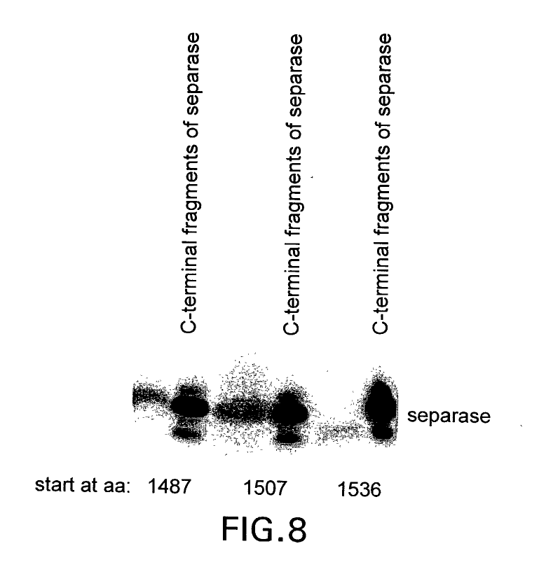

The PCR products were in vitro transcribed and translated with the TNT system (Promega). The recombinant products, which start with an exogenous methionine at the indicated amino acids obtained, were loaded side by side with in vitro cleaved separase on a SDS-PAGE. For immunoblotting the mouse anti-separase antibody (7A6) was used. The results are shown in FIG. 8.

### EXAMPLE 9

Assay for Identifying Separase Inhibitors Using a Fluorogenic Peptid Substrate

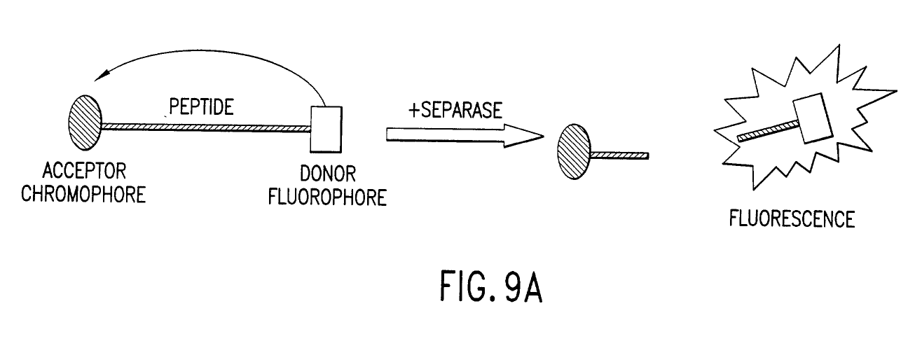

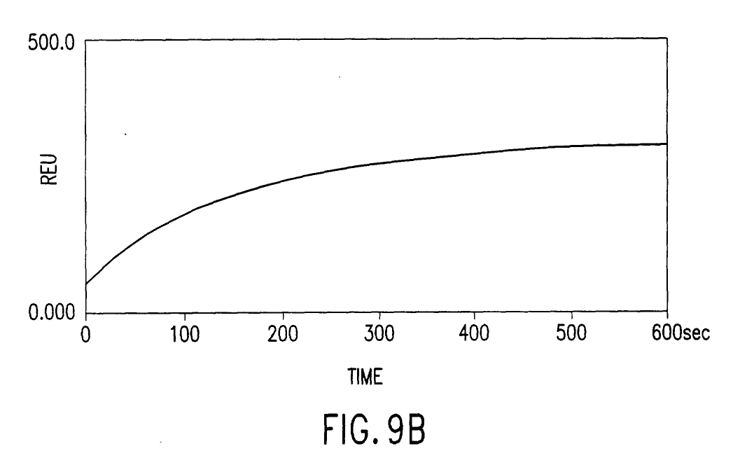

In order to establish a robust screening assay (based on liquid phase fluorescence energy transfer) for identifying inhibitors of recombinant human separase, four peptide substrates (1: SFEILR-AMC, 2: SFEILRG-AMC, 3:EWELLR-AMC and 4: DREIMR-AMC) were synthesized. These peptides are linked to AMC (7-Amido-4-methylcoumarin), a fluorogenic group, which has been described for proteolytic assays, such as for trypsin (Zimmerman et al., 1977) and cathepsin B (Barrett and Kirschke, 1981). AMC serves as a donor fluorophore and in the case of the separase-specific peptide substrates the amino acid bonds of the peptides function as acceptor chromophores (FIG. 9A). The peptide substrates are cleaved at the P1′-AMC junction; by processing the peptide-AMC bond the unquenched AMC is set free and can be monitored as increasing fluorescence. The designed peptide substrates represent the intramolecular cis-cleavage site in separase itself (peptides 1-3) and the intermolecular trans-cleavage site in cohesin respectively (peptide 4). Since all these peptides contain an Arg at the P1′ site, the peptides could be easily tested by utilizing trypsin (specific recognition site at P1′: Arg or Lys). Except for peptide 2, which contains an additional Gly between P1′ (Arg) and the AMC residue, all peptide substrates could be efficiently cleaved (FIG. 9B) by trypsin as follows:

Trypsin solution (Gibco 043-90317 FU) was diluted 1:1000 in Hepes buffer containing 20 mM Herpes (pH: 7.7), 100 mM KCI, 1 mM MgCl₂, 0.1 mM CaCl₂ and 1 mM DTT (freshly added). 1 μl of peptide 1 (4 mg/ml in DMSO) was added, mixed and measured in a Hitachi f-2000 fluorescence spectrophotometer (Ex: 355 and Em: 460 nm). A typical kinetics is shown in FIG. 9B.

Due to the complex activation procedure of recombinant human separase, a bead-suspension with coupled and activated separase had to be used for the establishment of a separase assay (activation of separase is described in Materials and Methods). The separase assay was performed as follows:

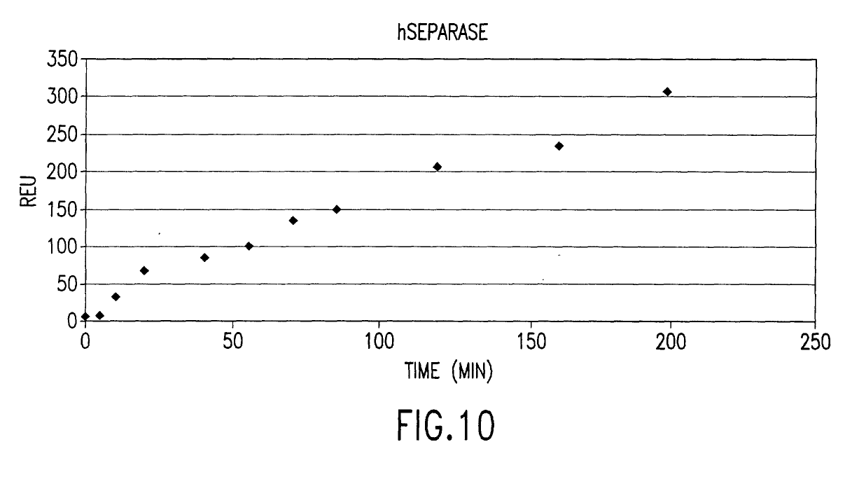

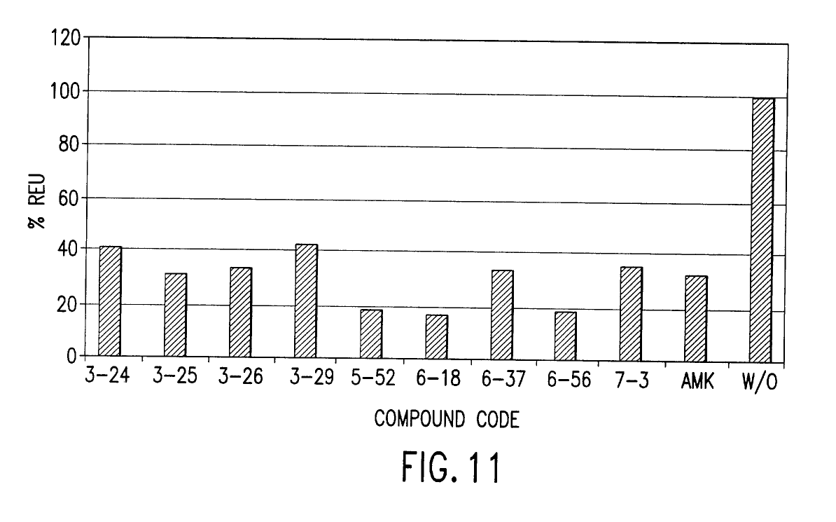

˜500 μl of separase bead suspension was diluted with 1800 μl Hepes buffer. 30 μl of the diluted suspension was applied per well of a 96 “Packard OptiPlate black” plate. Additionally, 70 μl Hepes buffer and 1 μl of a LMW-compound stock solution (5 mg/ml in DMSO) were added per well. After 10 minutes of pre-incubation at room temperature the reaction was initialized by adding 1 μl of the peptide 1 (4 mg/ml in DMSO). The reaction was monitored in a Fluoroskan II 96-well reader (Ex: 355 nm, Em: 460 nm) at room temperature (FIG. 10). As a control, measurement was performed in the presence of 2 μl DMSO per 100 μl reaction volume whereas for inhibition a cleavage peptide linked to an AMK (acyl-oxymethyl ketone) residue, which was described in WO 00/48627, was used. The AMK residue serves as a broad-spectrum inhibitor for many cysteine and serine proteases (for details see Beynon and Bond, 1989). In parallel the activity of the separase preparations bound to the beads was tested in an in vitro assay demonstrating the cleavage of cohesin (for details see Example 2, 3, FIG. 10). From a compound pool, 51 compounds that were shown to be potential protease inhibitors in preliminary experiments, were selected and tested for their ability to interfere with the activity of human separse. Out of these compounds, nine were found to inhibit separase activity within the same range as the AMK peptide (FIG. 11).

### REFERENCES

Ambrose W P, et al., (1998), Anal Biochem, October 15, 263(2): 150-7

Barrett and Kirschke, 1981, Meth. Enzymol. 80, 535

Beynon and Bond, “Proteolytic Enzymes, a Practical Approach”, IRL Press 1989

Brown A M, et al., (1994), Anal Biochem, February 15, 217(1): 139-47

Buonomo S B, Clyne R K, Fuchs J, Loidl J, Uhlmann F, Nasmyth K; Cell Oct. 27, 2000;103(3):387-98

Cerretani M, et al., (1999), Anal Biochem, January 15, 266(2): 192-7

Ciosk R, Zachariae W, Michaelis C, Shevchenko A, Mann M, Nasmyth K; Cell Jun. 12, 1998;93(6):1067-76

Elbashir et al., (2001) Nature 411(6836):494-8.

Faleiro L, Kobayashi R, Fearnhead H, Lazebnik Y; EMBO J May 1, 1997;16(9):2271-81

Gershkovich, A. A. and Kholodovych, V. V. (1996), J Biochem Biophys Meth 33, 135

Gmachl M, Gieffers C, Podtelejnikov A V, Mann M, Peters J M; Proc Natl Acad Sci USA Aug. 1, 2000;97(16):8973-8

Gray N M, et al., (1994), Anal Biochem, 216(1): 89-96

Hauf S., Waizenegger I C, Peters J M; Science Aug. 17, 2000; 293: 1320-1323

Jolley, M. E. (1996), J Biomol Screening 1, 33

Lee I A, Seong C, Choe I S; Biochem Mol Biol Int 1999 May;47(5):891-7

Levine L M, et al., (1997), Anal Biochem, April 5, 247(1): 83-8

Matayoshi, E. D. (1990), Science 247, 954

Murray M G, et al., (1993), Gene, November 30, 134(1):123-8

Nasmyth K, Peters J M, Uhlmann F; Science May 26, 2000;288(5470):1379-85

Remington's Pharmacuetical Sciences, 1980, Mack Publ. Co. Easton, Pa., Osol (ed.)

Sarubbi E, et al., (1991), FEBS Lett, February 25, 279(2): 265-9

Singh J, et al., (1996), Bioorg Med Chem, 4(5): 639-43

Stebbins J. and Debouck C., (1997), Anal Biochem, June 1, 248(2): 246-50

Taliani M, et al., (1996), Anal Biochem, August 15, 240(1):60-7

Uhlmann F, Wernic D, Poupart M A, Koonin E V, Nasmyth K; Cell Oct. 27, 2000;103(3):375-86

Uhlmann F, Lottspeich F, Nasmyth K; Nature Jul. 1, 1999;400(6739):37-42

Waizenegger I C, Hauf S, Meinke A, Peters; J M Cell Oct. 27, 2000;103(3): 399-410

Zhang X, Horwitz G A, Heaney A P, Nakashima M, Prezant T R, Bronstein M D, Melmed S; J Clin Endocrinol Metab 1999 Feb.;84(2):761-7

Zimmerman et al., 1977, Anal. Biochem. 78, 47

1. A method for identifying a compound that has the ability of modulating sister chromatid separation by inhibiting the proteolytic activity of separase, characterized in that an active separase in the form of
a) one or more separase fragment(s), optionally upon activation in the presence of securin, or
b) the full-length separase upon activation in the presence of securin,
is incubated in the presence of a separase substrate, with a test compound and that the modulating effect of the test compound on the proteolytic activity of the active separase is determined.

2. The method of claim 1, wherein the active separase is human.

3. The method of claim 1 or 2, wherein the active separase (fragment) is activated in a mitotic cell extract in the presence of securin.

4. The method of claim 3, wherein the mitotic cell extract has been obtained from Xenopus laevis eggs.

5. The method of claim, wherein the separase substrate is peptide that carries a fluorogenic group, which upon processing of the peptide results in a change in fluorescence and that the change in fluorescence is correlated with the separase activity.

6. The method of claim 5, wherein the separase substrate is a peptide selected from peptides containing the sequence DREIMR, SFEILR or EWELLR.

7. A peptide selected from peptides containing the sequence DREIMR, SFEILR or EWELLR or a derivate thereof.

8. The peptide of claim 7 or a derivate thereof for the treatment of cancer.

9. Pharmaceutical composition containing, as the active ingredient, the peptide(derivative) of claim 7.

10. An inhibitor of separase which has been identified in the method of claim 1 for human therapy.
